(19) United States
(12) Patent Application Publication
(10) Pub. No.: US 2005/0220758 A1
(43) Pub. Date: Oct. 6, 2005

(54) Lyophilised preparation comprising immunocytokines

(76) Inventors: Hans-Peter Zobel, Florsheim (DE); Sven Oliver Arndt, Zwingenberg (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/503,615

(22) PCT Filed: Jan. 14, 2003
(86) PCT No.: PCT/EP03/00259

§ 371 Date: Aug. 5, 2004

(30) Foreign Application Priority Data
Feb. 6, 2002 (DE) 102 04 792.8

(51) Int. Cl. A61K 38/20; A61K 31/7012; A61K 31/7008

(52) U.S. Cl. 424/85.1; 424/85.2; 514/53; 514/62

(57) ABSTRACT

The invention relates to a lyophilised pharmaceutical preparation comprising an immunocytokine. The preparation has an increased shelf life, even at elevated temperatures, and, after reconstitution, can be administered parenterally as a medicament.

The present invention relates to a stable lyophilised pharmaceutical preparation comprising immunocytokines and to the preparation of the lyophilised pharmaceutical preparation.

Immunocytokines are conjugates consisting of antibodies and cytokines, where the carboxyl-terminated ends of the two heavy immunoglobulin chains of the antibodies are each linked to the N-terminated ends of a cytokine.

Antibodies are certain glycoproteins having a protective action which occur in the blood, lymph and body secretions as a consequence of immunisation by antigens and undergo an antigen-antibody reaction therewith. Antibodies belong to the immunoglobulins (Ig) and can be sub-divided into 5 classes: IgA, IgD, IgE, IgG and IgM, some of which can in turn be sub-divided into further sub-classes (isotypes), for example into IgG1, IgG2, IgG3, IgG4, IgA and IgA2. The immunocytokines include all IgG antibodies. They cover monoclonal antibodies, polyclonal antibodies and multispecific antibodies, such as, for example, bispecific antibodies.

Cytokines are polypeptides which are excreted endocrinically or paracrinically, i.e. into the blood or the surrounding tissue, by cells and, after binding to specific receptors, influence the functions (usually division and growth, but also, for example, locomotion) of other cells. In some cases, the cytokine-producing cells are themselves the object of this regulation (then referred to as autocrinic). Cytokines regulate, inter alia, the complex interaction of the cells of the immune system.

Examples of cytokines are lymphokines, monokines and conventional polypeptide hormones. Cytokines include growth hormones, such as human growth hormone, human N-methionyl growth hormone and bovine growth hormone, parathormone, thyroxin, insulin, proinsulin, relaxin, prorelaxin, glycoprotein hormones, such as follicle-stimulating hormone (FSH), thyrotropin (TSH) and lutropin (LH), hepatic growth factor, fibroblast growth factor, prolactin, placenta lactogen, mouse gonadotrophin-associated peptide, inhibin, activin, vascular endothelial growth factor (VEGF), integrin, thrombopoietin (TPO), nerve growth factors, such as NGFβ, platelet growth factor, transforming growth factors (TGFs), such as TGFα and TGFβ, erythropoietin (EPO), interferons, such as IFNα, IFNβ and IFNγ, haematopoietic growth factors, such as M-CSF, GM-CSF and G-CSF, interleukins (ILs), such as IL-1, IL-1a, IL-2, IL-3, IL4, IL-5, IL-6, IL-7, IL-8, IL-9, IL-10, IL-11, IL-12, and tumour necrosis factors (TNFs), such as TNFα or TNFβ.

Like the above-mentioned antibodies and cytokines, immunocytokines are peptide active ingredients and therefore cannot be absorbed enterally. For therapeutic application, they therefore generally have to be administered parenterally in the form of a solution.

One problem in the formulation of solutions comprising peptide active ingredients is their tendency towards aggregation and towards the formation of protein multimers. However, this problem is of varying severity depending on the physicochemical properties of the particular active ingredients concerned. Whereas proteins having a hydrophilic character have a relatively low tendency towards the formation of aggregates in aqueous solution, proteins having a hydrophobic character have an increased tendency towards aggregation.

Antibodies consist of 2 anti-parallel folded sheets which are arranged in a sandwich-like manner to one another (conserved domains). Hydrophobic and hydrophilic amino acids alternate in the folded sheets, with the hydrophobic side chains of the two folded sheets in each case being directed towards one another and thus pointing into the interior of the sandwich structure, and the hydrophilic amino acids in each case pointing outward (J. Klein, Immunologie [Immunology], Verlag Chemie, Weinheim, 1991). The outward-pointing hydrophilic amino acids result in solubilisation of the antibodies in aqueous solution and thus prevent interaction between different antibodies. Antibodies thus have only low surface hydrophobicity and aggregation tendency.

Owing to the above-mentioned properties, solutions of antibodies are comparatively simple to formulate in a stable manner. One example of a commercially available product is Rituxan®, an aqueous formulation comprising the monoclonal antibody rituximab, an inorganic buffer and polysorbate. Removal of the water by freeze-drying enables the stability of the aqueous antibody solutions, which are already relatively stable per se, to be increased still further. Before administration, the lyophilisates obtained are then reconstituted by addition of water to the aqueous solution. An example of a product of this type is Remicade®, which, besides the monoclonal antibody infliximab, an inorganic buffer and a polysorbate, additionally comprises a sugar as freezing-protection agent or structure former.

WO 98/22136 A2 discloses a lyophilised preparation comprising an antibody, a sugar or amino sugar, an amino acid and a surfactant. Although the preparation is claimed for antibodies in general, only preparations comprising monoclonal antibodies which are directed against the hepatitis B virus (AK HBV) and in each case a preparation comprising an antibody against L-selectin (anti-L-selectin) and an antibody against the anti-L nerve growth factor receptor (anti-L-NGFR) are disclosed as working example.

Cytokines do not contain conserved domains which could cause good water solubility as in the case of antibodies. They therefore have an increased tendency towards aggregation in aqueous solution. This applies in particular to cytokines which contain a bundle of four α-helices as common structural feature (so-called 4 α-helix bundle cytokines) and have pronounced hydrophobicity owing to this structural feature. Hydrophobic interactions which accompany hydrophobicity are in turn often the cause/mechanism of aggregation (Hora-MS and Chen-B, (1999), Biopharm. Ind. Perspect., 217-248). Cytokines which contain a bundle of four α-helices as common structural feature are many interleukins, in particular IL-2, IL-3, IL4, IL-5, IL-6, IL-7, IL-10, IL-11 and IL-12, interferons, in particular IFNβ and IFNγ, haematopoietic growth factors, such as M-CSF, GM-CSF, G-CSF, erythropoietin (EPO) and stem cell factor (SCF). Cytokines which are explicitly described as hydrophobic in the literature are, for example, IL-2 (Robb-RJ et al. (1983) PNAS 80:5990-4; U.S. Pat. No. 5,580,856), IL4 (Sharma-S et al. (1987) 235:1489-92), IL-5 (Takatsu-K et al. (1985) JI 134:382-9), G-CSF (U.S. Pat. No. 5,104,65).

The strong tendency of cytokines, in particular the 4 α-helix bundle cytokines, requires particular measures for their stabilisation. For example, the product Proleukin®, a lyophilisate comprising the active ingredient interleukin-2, comprises as adjuvants a sugar, an inorganic buffer and an anionic detergent (sodium lauryisulfate). Anionic detergents, in particular if the medicament is intended for parenteral administration, are, however, extremely dubious from a toxicological point of view.

A further example which indicates the particular difficulties in the stabilisation of cytokine-containing formulations are the products on the market comprising interferon β (Avonex®, Betaferon®, Rebif®). All these products are stabilised by albumin, which should likewise be classified as extremely critical from a toxicological point of view, in particular with respect to undesired immune reactions.

Owing to the above-mentioned differences between cytokines and antibodies, the immunocytokines, which are each composed of one antibody and two cytokines, also differ significantly in their physicochemical properties from those of the antibodies. In particular, immunocytokines containing a cytokine having four α-helix bundles have a strong tendency towards the formation of agglomerates in aqueous owing to the associated pronounced hydrophobicity and are difficult to stabilise.

The object of the present invention was to provide a stabilised preparation for immunocytokines. The preparation should not comprise any toxicologically unacceptable adjuvants, should be stable for an extended period under increased stress conditions, such as elevated temperature and atmospheric humidity, and should be reconstitutable with an aqueous solvent to give a ready-to-administer solution having a high active-ingredient content.

Surprisingly, it has been possible to provide a preparation which meets these requirements by freeze-drying an aqueous buffered solution which, besides an immunocytokine, comprises a sugar or an amino sugar, an amino acid and a surfactant. The present invention therefore relates to a stable lyophilised preparation comprising an immunocytokine, a sugar or an amino sugar, an amino acid and a surfactant.

The preparation preferably comprises an immunocytokine which, as cytokine constituent, cytokines selected from the group consisting of cytokines which have, as common structural feature, a bundle of four α-helices, in particular an interleukin, preferably IL-2, IL-3, IL4, IL-5, IL-6, IL-7, IL-10, IL-11 and/or IL-12, an interferon, preferably IFNβ and/or IFNγ, and/or a haematopoietic growth factor, preferably M-CSF, GM-CSF, G-CSF, EPO or SCF. The composition particularly preferably comprises an immunocytokine containing interleukin-2 (IL-2).

The preparation according to the invention is physiologically well tolerated, can be prepared easily, can be dispensed precisely and is stable with respect to assay, decomposition products and aggregates over the duration of storage and even after repeated freezing and thawing processes. It is stable on storage over a period of at least three months up to a period of two years at refrigerator temperature (2-8° C.) and at room temperature (23-27° C., 60% relative atmospheric. humidity (r.h.). Surprisingly, the preparation according to the invention is also stable on storage over the said period at elevated temperatures and higher atmospheric humidity levels, for example at a temperature of 40° C. and 75% r.h.

The lyophilised preparation can be reconstituted in a simple manner to give a ready-to-administer particle-free solution by addition of an aqueous solvent, for example water for injection purposes or an isotonic aqueous solution. The reconstituted solution is stable over a period of about 5 days, but is particularly preferably administered within 24 hours.

Reconstitution of the preparation according to the invention with aqueous solvents advantageously enables the preparation of immunocytokine-containing solutions having a pH of from 5 to 8, preferably having a pH of from 5.6 to 7.4, particularly preferably having a pH of 6-7, and an osmolality of from 250 to 350 mOsmol/kg. The reconstituted preparation can thus be administered directly intravenously, intraarterially and also subcutaneously, substantially without pain. In addition, the preparation can also be added to infusion solutions, such as, for example, glucose solution, isotonic saline solution or Ringer solution, which may also comprise further active ingredients, thus also enabling relatively large amounts of active ingredient to be administered.

According to a preferred embodiment of the invention, the lyophilised pharmaceutical preparation essentially consists of an immunocytokine, a sugar or amino sugar, an amino acid, a buffer and a surfactant.

The preparation according to the invention enables the preparation of immunocytokine solutions which are matched in their concentration to the clinical needs. Preference is given to immunocytokine solutions having an immunocytokine concentration of from about 0.1 to 25 mg/ml, particularly preferably from 1 to 10 mg/ml, very particularly preferably from 1 to 5 mg/ml.

The sugar employed in the preparation according to the invention can be mono-, di- or trisaccharides. These sugars can be employed either alone or in mixtures with sugar alcohols (for example mannitol). Examples of monosaccharides that may be mentioned are glucose, mannose, galactose, fructose and sorbose, examples of disaccharides that may be mentioned are sucrose, lactose, maltose or trehalose, and an example of a trisaccharide that may be mentioned is raffinose. Preference is given to sucrose, lactose, maltose or trehalose, particularly preferably sucrose and maltose.

It is also possible for amino sugars to be present, i.e. monosaccharides which contain a primary, secondary or tertiary amino group or an acylated amino group (—NH—CO—R) instead of a hydroxyl group. For the purposes of the invention, particular preference is given here to glucosamine, N-methyl-glucosamine, galactosamine and neuraminic acid.

The sugar/amino sugar present is present in the preparation according to the invention in such an amount that it is present in the resultant solution after reconstitution with the proposed volume of solvent in a concentration of from about 1 to 200 mg/ml. The sugar is preferably present in the reconstituted solution in a concentration of from 15 to 30 mg/ml.

Suitable amino acids used in accordance with the invention are basic, acidic or neutral amino acids, for example arginine, histidine, omithine, lysine, glycine, inter alia. The amino acids are preferably employed in the form of their inorganic salts (advantageously in the form of the hydrochloric acid salts, i.e. as amino acid hydrochlorides). In the case where the free amino acids are employed, the desired pH is set by addition of a suitable physiologically tolerated buffer substance, such as, for example, an organic or inorganic acid, such as citric acid and phosphoric acid, sulfuric acid, acetic acid, formic acid or salts thereof. Preference is given to citrates and phosphates, with which particularly stable lyophilisates are obtained.

Preferred amino acids are arginine, lysine and omithine. In addition, it is also possible to use acidic amino acids, such as, for example, glutamic acid and aspartic acid, or neutral amino acids, such as, for example, isoleucine, leucine and alanine, or aromatic amino acids, such as, for example, phenylalanine, tyrosine or tryptophan. The amino acid content in the preparation according to the invention is from 1 to 200 mmol/l, preferably from 40 to 100 mmol/l, particularly preferably 40-80 mmol/l (in each case based on the reconstituted solution).

Surfactants which can be employed are all surfactants usually used in pharmaceutical preparations, preferably nonionic surfactants, in particular polysorbates and polyoxyethylene-polyoxypropylene polymers. Particular preference is given to polyoxyethylene sorbitan fatty acid esters, in particular polyoxyethylene (20) sorbitan monolaurate and polyoxyethylene (20) sorbitan monooleate. In accordance with the invention, the preparation comprises from 0.001 to 1% by weight, preferably from 0.005 to 0.5% by weight and particularly preferably from 0.01 to 0.15% by weight (in each case based on the reconstituted solution).

If the preparation according to the invention comprises buffers, these can in principle be any physiologically tolerated substances which are suitable for setting the desired pH. The amount of buffer substance is selected in such a way that, after reconstitution-of the lyophilised preparation, for example with water for injection purposes, the resultant aqueous solution has a buffer concentration of from 5 mmol/l to 50 mmol/l, preferably from 10 to 20 mmol/l. Preferred buffers are citrate buffers or phosphate buffers. Suitable phosphate buffers are solutions of the mono- and/or disodium and potassium salts of phosphoric acid, such as disodium hydrogenphosphate or potassium dihydrogenphosphate, as well as mixtures of the sodium and potassium salts, such as, for example, mixtures of disodium hydrogenphosphate and potassium dihydrogenphosphate.

If the reconstituted solution is not already isotonic through the osmotic properties of the immunocytokine and through the adjuvants employed for stabilisation, an isotonic agent, preferably a physiologically tolerated salt, such as, for example, sodium chloride or potassium chloride, or a physiologically tolerated polyol or a sugar, such as, for example, glucose, glycerol or mannitol, may furthermore be present in an amount necessary for establishing isotonicity.

In addition, the lyophilisates according to the invention may comprise further physiologically tolerated adjuvants, such as, for example, antioxidants, such as ascorbic acid or glutathione, preservatives, such as phenol, m-cresol, methyl- or propylparaben, chlorobutanol, thiomersal or benzalkonium chloride, or further stabilisers, structure formers and solubilisers, such as polyethylene glycols (PEG), for example PEG 3000, 3350, 4000 or 6000, or cyclodextrins, for example hydroxypropyl-β-cyclodextrin, sulfo-butylethyl-β-cyclodextrin or γ-cyclodextrin, or dextrans.

The preparation according to the invention can be prepared by preparing an aqueous preparation comprising an immunocytokine as active ingredient and a sugar or amino sugar, an amino acid and a surfactant and, if desired, further pharmaceutical adjuvants, and subsequently lyophilising the solution.

The aqueous preparation can be prepared by adding the said adjuvants to a solution comprising an immunocytokine. To this end, defined volumes of stock solutions comprising the said further adjuvants in defined concentration are advantageously added to a solution having a defined concentration of immunocytokine, as obtained from its preparation, and the mixture is, if desired, diluted to the pre-calculated concentration with water. Alternatively, the adjuvants can also be added as solids to the starting solution comprising the immunocytokine. If the immunocytokine is in the form of a solid, for example in the form of a lyophilisate, the preparation according to the invention can be prepared by firstly dissolving the respective immunocytokine in water or an aqueous solution comprising one or more of the further adjuvants, and subsequently adding the amounts required in each case of stock solutions comprising the further adjuvants, the further adjuvants in solid form and/or water. The immunocytokine can advantageously also be dissolved directly in a solution comprising all further adjuvants. One or more of the adjuvants present in the preparation according to the invention may advantageously already have been added during or at the end of the process for the preparation of the particular immunocytokine. This can preferably be carried out in the final step of the purification carried out after its preparation by dissolving the immunocytokine directly in an aqueous solution comprising one, more than one or all of the further adjuvants or rebuffering it by suitable methods, such as tangential flow filtration. In order to prepare the preparation, the respective further ingredient(s) then need only be added in a smaller amount in each case and/or not added at all. It is particularly preferred for the respective ingredient to be dissolved directly in an aqueous solution comprising all further adjuvants in the final step of the purification carried out after its preparation, directly giving the solution to be lyophilised.

The solution comprising the respective immunocytokine and the adjuvants is set to a pH of from 5 to 8, sterile-filtered and freeze-dried.

The lyophilised preparation obtained can be reconstituted by addition of an aqueous solvent to give an aqueous preparation which can be administered directly, in particular parenterally. The present invention therefore also relates to an aqueous pharmaceutical preparation of immunocytokines which is obtainable by-reconstitution of the lyophilisate according to the invention with an aqueous solvent.

The reconstituted aqueous pharmaceutical preparation preferably has a pH of 5-8, preferably a pH of 5.6-7.4 and particularly preferably a pH of 6.0-7.0.

The examples explain the invention without being restricted thereto.

### EXAMPLE 1

### Batch 8020

Lyophilisate from Aqueous Solution Comprising:
0.7 mg/ml of EMD 273066 (huKS-IL2)
5 mmol/l of citric acid
100 mmol/l of arginine HCl
1.5% by weight of sucrose
0.01% by weight of polyoxyethylene (20) sorbitan monooleate (Tween 80)

The preparation was carried out by mixing defined volumes of aqueous solutions comprising the respective adjuvants in defined concentration. The following solutions were used:

Solution A (Active-Ingredient Solution) Comprising:
5 mg/ml of EMD 273066
5 mmol/l of citric acid
100 mmol/I of arginine HCl
0.01% by weight of polyoxyethylene (20) sorbitan monooleate (Tween 80)
NaOH q.s. to pH 7.0

Solution B (Adjuvant Solution):
1.744% by weight of sucrose
5 mmol/l of citric acid
100 mmol/l of arginine HCl
0.01% by weight of polyoxyethylene (20) sorbitan monooleate (Tween 80)
NaOH q.s. to pH 7.0

In order to prepare the preparation according to the invention, 100 ml of solution A and 614 ml of solution B were combined with one another.

The prepared solution was sterile-filtered before packaging. 6 ml vials were each filled with 4 ml of solution. The vials were subsequently pre-sealed with stoppers and lyophilised. After freeze-drying, the vials were sealed and crimped.

### EXAMPLE 2

### Batch 8021

Lyophilisate from Aqueous Solution Comprising:
0.7 mg/ml of EMD 273066 (huKS-IL2)
5 mmol/l of citric acid
100 mmol/l of arginine HCl
1.5% by weight of maltose
0.01% by weight of polyoxyethylene (20) sorbitan monooleate (Tween 80)

The preparation was carried out by mixing defined volumes of aqueous solutions comprising the respective adjuvants in defined concentration. The following solutions were used:

Solution A (Active-Ingredient Solution) Comprising:
5 mg/ml of EMD 273066
5 mmol/l of citric acid
100 mmol/l of arginine HCl
0.01% by weight of polyoxyethylene (20) sorbitan monooleate (Tween 80)
NaOH q.s. to pH 7.0

Solution B (Adjuvant Solution):
1.744% by weight of maltose
5 mmol/l of citric acid
100 mmol/l of arginine HCl
0.01% by weight of polyoxyethylene (20) sorbitan monooleate (Tween 80)
NaOH q.s. to pH 7.0

In order to prepare the preparation according to the invention, 100 ml of solution A and 614 ml of solution B were combined with one another.

The prepared solution was sterile-filtered before packaging. 6 ml vials were each filled with 4 ml of solution. The vials were subsequently pre-sealed with stoppers and lyophilised. After freeze-drying, the vials were sealed and crimped.

### EXAMPLE 3

### Batch 8431

Lyophilisate from Aqueous Solution Comprising:
1 mg/ml of EMD 273066 (huKS-IL2)
5 mmol/l of citric acid
100 mmol/l of arginine HCl
1.5% by weight of sucrose
0.01% by weight of polyoxyethylene (20) sorbitan monooleate (Tween 80)

The preparation was carried out by mixing defined volumes of aqueous solutions comprising the respective adjuvants in defined concentration. The following solutions were used:

Solution A (Active-Ingredient Solution) Comprising:
1.45 mg/ml of EMD 273066
1.5% by weight of sucrose
5 mmol/l of citric acid
NaOH q.s. to pH 7.0

Solution B (Adjuvant Solution):
1.5% by weight of sucrose
5 mmol/l of citric acid
287 mmol/l of arginine HCl
0.0283% by weight of polyoxyethylene (20) sorbitan monooleate (Tween 80)
NaOH q.s. to pH 7.0

In order to prepare the preparation according to the invention, 46.9 ml of solution A and 25.1 ml of solution B were combined with one another.

The prepared solution was sterile-filtered before packaging. 6 ml vials were each filled with 2 ml of solution. The vials were subsequently pre-sealed with stoppers and lyophilised. After freeze-drying, the vials were sealed and crimped.

### EXAMPLE 4

### Batch 8591

Lyophlisate from Aqueous Solution Comprising:
4 mg/ml of EMD 273066 (huKS-IL2)
12.5 mmol/l of citric acid
80 mmol/l of arginine HCl
1.8% by weight of sucrose
0.008% by weight of polyoxyethylene (20) sorbitan monooleate (Tween 80)

The preparation was carried out by mixing defined volumes of aqueous solutions comprising the respective adjuvants in defined concentration. The following solutions were used:

Solution A (Active-Ingredient Solution) Comprising:
5 mg/ml of EMD 273066
5 mmol/l of citric acid
100 mmol/l of arginine HCl
0.01% by weight of polyoxyethylene (20) sorbitan monooleate (Tween 80) NaOH q.s. to pH 6.0

Solution B (Adjuvant Solution):
8.7% by weight of sucrose
41 mmol/l of citric acid
NaOH q.s. to pH 6.0

In order to prepare the preparation according to the invention, 4 ml of solution A and 15.5 ml of solution B were combined with one another.

The prepared solution was sterile-filtered before packaging. 2 ml vials were each filled with 1 ml of solution. The vials were subsequently pre-sealed with stoppers and lyophilised. After freeze-drying, the vials were sealed and crimped.

### EXAMPLE 5

### Comparative Formulation 1 with Mannitol Instead of Sucrose, Batch 8008

Lyophilisate from Aqueous Solution Comprising:
0.7 mg/ml of EMD 273066 (huKS-IL2)
5 mmol/l of citric acid
100 mmol/l of arginine HCl
4% by weight of mannitol
0.01% by weight of polyoxyethylene (20) sorbitan monooleate (Tween 80) to pH 7.0 with NaOH

The preparation was carried out by direct freeze-drying of the active-ingredient solution having the above-mentioned composition.

The prepared solution was filtered using a sterile filter before packaging. 6 ml vials were filled with 4 ml of solution. The vials were subsequently pre-sealed with stoppers and lyophilised. After freeze-drying, the vials were sealed and crimped.

### EXAMPLE 6

### Comparative Preparation 2, Batch 8434, Corresponds in Composition to Batch 8431 Without Addition of Arginine

Lyophilisate from Aqueous Solution Comprising:
1 mg/ml of EMD 273066 (huKS-IL2)
5 mmol/l of citric acid
1.5% by weight of sucrose
0.01% by weight of polyoxyethylene (20) sorbitan monooleate (Tween 80)

The preparation was carried out by mixing defined volumes of aqueous solutions comprising the respective adjuvants in defined concentration. The following solutions were used:

Solution A (Active-Ingredient Solution) Comprising:
1.45 mg/ml of EMD 273066
1.5% by weight of sucrose
5 mmol/l of citric acid
NaOH q.s. to pH 7.0

Solution B (Adjuvant Solution):
1.5% by weight of sucrose
5 mmol/l of citric acid
0.0283% by weight of polyoxyethylene (20) sorbitan monooleate (Tween 80)
NaOH q.s. to pH 7.0

In order to prepare the preparation according to the invention, 46.9 ml of solution A and 25.1 ml of solution B were combined with one another.

The prepared solution was sterile-filtered before packaging. 6 ml vials were each filled with 2 ml of solution. The vials were subsequently pre-sealed with stoppers and lyophilised. After freeze-drying, the vials were sealed and crimped.

### EXAMPLE 7

### Comparative Preparation 3, Batch 8430, Corresponds in Composition to Batch 8431 Without Addition of Tween 80

Lyophilisate from Aqueous Solution Comprising:
1 mg/ml of EMD 273066 (huKS-IL2)
5 mmol/l of citric acid
100 mmol/l of arginine HCl
1.5% by weight of sucrose

The preparation was carried out by mixing defined volumes of aqueous solutions comprising the respective adjuvants in defined concentration. The following solutions were used:

Solution A (Active-Ingredient Solution) Comprising:
1.45 mg/ml of EMD 273066
1.5% by weight of sucrose
5 mmol/l of citric acid
NaOH q.s. to pH 7.0

Solution B (Adjuvant Solution):
1.5% by weight of sucrose
5 mmol/l of citric acid
287 mmol/l of arginine HCl
NaOH q.s. to pH 7.0

In order to prepare the preparation according to the invention, 46.9 ml of solution A and 25.1 ml of solution B were combined with one another.

The prepared solution was sterile-filtered before packaging. 6 ml vials were each filled with 2 ml of solution. The vials were subsequently pre-sealed with stoppers and lyophilised. After freeze-drying, the vials were sealed and crimped.

### EXAMPLE 8

### Comparative Preparation 4, Batch 8429, Corresponds in Composition to Batch 8431 Without Addition of Tween 80 and Without Addition of Arginine

Lyophilisate from Aqueous Solution Comprising:
1 mg/ml of EMD 273066 (huKS-IL2)
5 mmol/l of citric acid
1.5% by weight of sucrose

The preparation was carried out by mixing defined volumes of aqueous solutions comprising the respective adjuvants in defined concentration. The following solutions were used:

Solution A (Active-Ingredient Solution) Comprising:
1.45 mg/ml of EMD 273066
1.5% by weight of sucrose
5 mmol/l of citric acid
NaOH q.s. to pH 7.0

Solution B (Adjuvant Solution):
1.5% by weight of sucrose
5 mmol/l of citric acid
NaOH q.s. to pH 7.0

In order to prepare the preparation according to the invention, 46.9 ml of solution A and 25.1 ml of solution B were combined with one another.

The prepared solution was sterile-filtered before packaging. 6 ml vials were each filled with 2 ml of solution. The vials were subsequently pre-sealed with stoppers and lyophilised. After freeze-drying, the vials were sealed and crimped.

Investigations of the Stability of the Preparations

The stability of the preparations according to the invention was tested in stability studies. To this end, the lyophilisates prepared were stored at various temperatures for certain times and investigated using suitable analytical methods. The climatic conditions of 40° C. with a relative atmospheric humidity (r.h.) of 75% were selected as stress condition in order to achieve differences in stability rapidly in the various formulations. Possible instabilities are evident in immunocytokines principally from the formation of aggregates and from the formation of degradation products. Degradation products and soluble aggregates are preferably determined by size exclusion chromatography (HPLC-SEC), while visual inspection and turbidity measurements serve for the detection of visible aggregates. The ELISA test likewise employed for evaluation of the preparations serves for testing the integrity and binding ability to the receptor. Together with UV photometry at a wavelength of 280 nm, it additionally serves for determination of the content.

Analytical Test Methods:

Appearance

The formulations prepared were investigated visually with the aid of a cold-light source for particles and for the occurrence of possible turbidity.

Protein Concentration: A₂₈₀ nm

The absorption of the resultant protein solutions at a wavelength of 280 nm was employed for determining the concentration of the preparations prepared. The extinction coefficient of 1.41 for the active ingredient huKS-IL2 (EMD 273066) was determined by quantitative amino-acid analysis. For the actual measurement, the protein solutions were diluted in triplicates until the absorption of the test solutions was between 0.1 and 1.0 (corresponding to a protein concentration of 0.5 mg/ml) absorption units. The absorption of the active ingredient-containing test solutions was measured against a corresponding reference solution containing no active ingredient.

Purity: Size Exclusion Chromatography, SEC-HPLC

Size exclusion chromatography (SEC) is an analytical method with which the purity and monomer/aggregate proportion of the preparations prepared can be determined. The components of the test solutions are separated on the basis of their molecular size via a special porous HPLC column. Relatively large molecules are eluted together with the exclusion volume, while relatively small molecules penetrate into the pores of the stationary phase to various extents and are retained thereby more or less strongly. Relatively small molecules, such as degradation products of huKS-IL2, therefore appear at later retention times than huKS-IL2 monomers and particularly than huKS-IL2 aggregates, which are the first to be eluted from the column.

Concentration and Integrity of the Active-Ingredient Molecule: KSA-ELISA

In this analytical method (ELISA, enzyme linked immunosorbent assay), microtitre plates are coated with the specific antigen for huKS-IL2 (EPCAM or KSA antigen). huKS-IL2 molecules in the test solution to be determined bind to the antigen via their antibody component and are thus bound to the microtitre plate. After addition of biotinylated anti-IL2 antiserum, the anti-IL2 antibodies react with IL2 moieties of the bound huKS-IL2 molecules. Excess anti-IL2 molecules are removed by washing the microtitre plate. Added streptavidin peroxidase conjugate is bound via the biotin and oxidises the leuko form of the dye tetramethylbenzidine (TMB) added in a later step to give the blue dye. The oxidation reaction is stopped after a defined time by addition of-phosphoric acid. A yellow coloration of the solution occurs, which can be quantified at a wavelength of 450 nm. The concentration of the protein test solutions here is proportional to the absorption determined at this wavelength.

Results:

The results in Tables 1 and 2 (batches 8020 and 8021) clearly confirm the quality and stability of the preparations prepared. The climatic conditions of 40° C. with a relative atmospheric humidity (r.h.) of 75% were selected as stress condition in order to achieve differences in stability rapidly in the various formulations. Surprisingly, the above-mentioned preparations are stable even at elevated storage temperatures and elevated relative atmospheric humidity (40° C./75% r.h.) over a period of >6 months. A corresponding comparative-formulation 1 (Table 3, batch 8008, Example 5), in which the sugar alcohol mannitol was employed as structure former instead of the disaccharides (sucrose or maltose), had already aggregated after 4 weeks under the corresponding stress condition. After storage for 26 weeks at room temperature (25° C., 60% r.h.), visible aggregates had likewise permeated the corresponding climatic pattern. Comparative formulation 1 can thus only be stored with cooling, in contrast to the preparation according to the invention.

Example formulation 4 (batch 8591) is a further example of the excellent stability of the preparation according to the invention and also shows that the formulation can also be applied to increased protein concentrations (see Table 4). This formulation was stored at a temperature of 40° C. (75% r.h.) for a duration of 14 weeks.

In a further series of experiments, it was tested whether all adjuvants in the preparations according to the invention are actually necessary for stabilisation. The experimental batch 8431 here comprised all constituents of the formulation according to the invention, while the comparative formulations lacked individual components:
formulation in accordance with Example 3 (batch 8431): all components are present
comparative formulation 2 (Example 6, batch 8434): without addition of arginine
comparative formulation 3 (Example 7, batch 8430): without addition of Tween 80
comparative formulation 4 (Example 8, batch 8429): without addition of Tween 80 and arginine

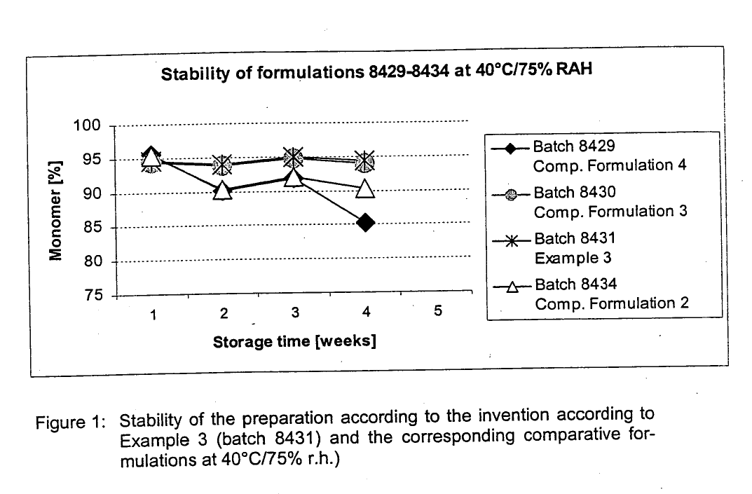

The result shown in FIG. 1 clearly confirms that the addition of the amino acid is absolutely necessary for the stability of the preparation according to the invention. According to this figure, although the addition of Tween 80 does not appear to be necessary, this does not entirely correspond to the facts. Tween 80 is added as early as during protein purification during preparation of the active-ingredient solutions in order to prevent the formation of, in particular, visible aggregates. The Tween 80 concentration employed here is above the critical micelle concentration (the CMC for Tween 80 is 0.001%). In the case of the series of experiments, it was attempted to remove both arginine and Tween by the so-called tangential flow filtration method. In this, a buffer exchange was carried out by means of diafiltration via a 50 kD membrane. However, Tween 80 micelles are in some cases above the membrane exclusion limit and are therefore not removed completely.

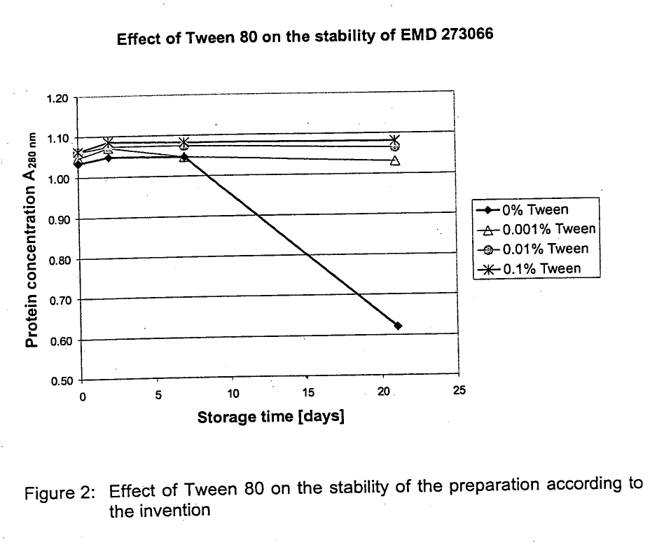

The addition of Tween 80 to the preparation according to the invention is absolutely necessary for re-dissolution of the lyophilisates and for the stability of the solution obtained after reconstitution, as a further study confirms. Starting from an EMD 273066 (huKS-IL2) active-ingredient solution comprising Tween 80, the adjuvant Tween 80 was separated off here by affinity chromatography via a protein A column. Tween 80 was added in increasing amounts to the Tween 80-free solution obtained. The preparations obtained were stressed for a period of 21 days at a temperature of 25° C. in 2 ml vials with the aid of a laboratory shaker. The stressed formulations were checked visually daily and analysed photometrically for their protein content at certain times. The result of this study is shown in FIG. 2. In the case of the preparations without addition of Tween 80, slight turbidity was observed after only one day, which became increasingly visible over the course of time. After a period of 21 days, a considerable drop in content was also noted, as shown in the figure.

TABLE 1
Stability of the preparation 8020 according to Example 1

| | | | | Appearance of | |
|---|---|---|---|---|---|
| Example 1 | | | | lyophilisate after | |
| Storage | | | | dissolution in | |
| time | Protein | HPLC- | KSA- | 4.0 ml of bidist. | Turbidity by |
| (weeks) | determination | SEC | ELISA | water | photometry |
| [weeks] | [mg/ml] | [%] | [mg/ml] | dissolved | A550 |
| Starting | 0.798 | 96.83 | 0.870 | — | 0.0036 |
| solution | | | | | |
| Lyophilisate | 0.849 | 96.68 | 0.857 | clear soln. slightly | 0.0033 |
| after prepn. | | | | opalescent | |

FREEZER
Storage at −20° C.

| | | | | | |
|---|---|---|---|---|---|
| 4 | — | 96.72 | — | clear colourless | 0.0019 |
| | | | | solution | |
| 8 | — | 96.89 | — | — | — |
| 13 | — | 97.02 | — | clear colourless | — |
| | | | | solution | |
| 26 | 0.808 | 97.38 | 0.685 | clear colourless | 0.0021 |
| | | | | solution | |

REFRIGERATOR
Storage at 2-8° C.

| | | | | | |
|---|---|---|---|---|---|
| 4 | — | 96.70 | | clear colourless | 0.0015 |
| | | | | solution | |
| 8 | — | 97.01 | | — | — |
| 13 | — | 97.01 | | clear colourless | — |
| | | | | solution | |
| 26 | 0.806 | 97.25 | 0.735 | clear colourless | 0.0011 |
| | | | | solution | |

Storage at 25° C./60% r.h.

| | | | | | |
|---|---|---|---|---|---|
| 4 | — | 96.66 | — | clear colourless | 0.0011 |
| | | | | solution | |
| 8 | — | 97.06 | 0.757 | — | — |
| 13 | — | 96.93 | — | clear colourless | — |
| | | | | solution | |
| 26 | 0.809 | 97.23 | 0.720 | clear colourless | 0.0017 |
| | | | | solution | |

Storage at 40° C./75% r.h.

| | | | | | |
|---|---|---|---|---|---|
| 4 | — | 96.62 | — | clear colourless | 0.0013 |
| | | | | solution | |
| 8 | — | 97.00 | 0.827 | — | — |
| 13 | — | 96.99 | — | clear colourless | — |
| | | | | solution | |
| 26 | 0.816 | 97.19 | 0.775 | clear colourless | 0.0007 |
| | | | | solution | |

TABLE 2
Stability of the preparation 8021 according to Example 2

| | | | | Appearance of | |
|---|---|---|---|---|---|
| Example 2 | | | | lyophilisate after | |
| Storage | | | | dissolution in | |
| time | Protein | HPLC- | KSA- | 4.0 ml of bidist. | Turbidity by |
| (weeks) | determination | SEC | ELISA | water | photometry |
| [weeks] | [mg/ml] | [%] | [mg/ml] | dissolved | A550 |
| Starting | 0.810 | 96.87 | 0.865 | — | 0.0042 |
| solution | | | | | |
| Lyophilisate | 0.868 | 96.70 | 0.817 | clear solution | 0.0034 |
| after prepn. | | | | | |

FREEZER
Strorage at −20° C.

| | | | | | |
|---|---|---|---|---|---|
| 4 | — | 97.12 | — | clear colourless | 0.0013 |
| | | | | solution | |
| 8 | — | 97.19 | — | — | — |
| 13 | — | 97.11 | — | clear colourless | — |
| | | | | solution | |
| 26 | — | — | — | — | |

REFRIGERATOR
Storage at 2-8° C.

| | | | | | |
|---|---|---|---|---|---|
| 4 | — | 97.27 | | clear colourless | 0.0018 |
| | | | | solution | |
| 8 | — | 97.07 | | — | — |
| 13 | — | 97.07 | | clear colourless | — |
| | | | | solution | |
| 26 | — | — | — | — | — |

Storage at 25° C./60% r.h.

| | | | | | |
|---|---|---|---|---|---|
| 4 | — | 97.27 | — | clear colourless | 0.0015 |
| | | | | solution | |
| 8 | — | 97.14 | 0.753 | — | — |
| 13 | — | 96.96 | — | clear colourless | — |
| | | | | solution | |
| 26 | — | — | — | — | — |

Storage at 40° C./75% r.h.

| | | | | | |
|---|---|---|---|---|---|
| 4 | — | 97.17 | — | clear colourless | 0.0013 |
| | | | | solution | |
| 8 | — | 97.21 | 0.820 | — | — |
| 13 | — | 96.91 | — | clear colourless | — |
| | | | | solution | |
| 26 | | | | | |

TABLE 3
Stability of the comparative preparation 1 (batch 8008 according to
Example 5)

| | | | | Appearance of | |
|---|---|---|---|---|---|
| Batch: 008008 | | | | lyophilisate after | |
| Storage | | | | dissolution in | |
| time | Protein | HPLC- | KSA- | 4.0 ml of bidist. | Turbidity by |
| (weeks) | determination | SEC | ELISA | water | photometry |
| [weeks] | [mg/ml] | [%] | [mg/ml] | dissolved | A550 |
| Lyophilisate | 0.763 | 94.79 | 0.667 | clear solution | 0.0033 |
| after prepn. | | | | | |

FREEZER
Storage at −20° C.

| | | | | | |
|---|---|---|---|---|---|
| 4 | — | — | — | — | — |
| 8 | — | — | — | — | — |
| 21 | — | 96.63 | — | clear colourless | 0.0035 |
| | | | | solution | |
| 26 | 0.766 | 96.16 | — | clear colourless | 0.0040 |
| | | | | solution | |

REFRIGERATOR
Storage at 2-8° C.

| | | | | | |
|---|---|---|---|---|---|
| 4 | — | — | — | clear colourless | — |
| | | | | solution | |
| 8 | — | — | — | — | — |
| 21 | — | 96.42 | | clear colourless | 0.0037 |
| | | | | solution | |
| 26 | 0.806 | 95.74 | — | clear colourless | 0.0043 |
| | | | | solution | |

Storage at 25° C./60% r.h.

| | | | | | |
|---|---|---|---|---|---|
| 4 | — | — | — | clear colourless | — |
| | | | | solution | |
| 8 | | — | — | clear colourless | — |
| | | | | solution | |
| 21 | — | 91.34 | — | slightly opalesc. | 0.0064 |
| | | | | colourless soln. | |
| 26 | — | 81.33 | — | slightly turbid, | 0.0209 |
| | | | | milky solution | |

Storage at 40° C./75% r.h.

| | | | | | |
|---|---|---|---|---|---|
| 4 | — | — | — | turbid solution | — |
| 8 | — | — | — | turbid solution | — |
| 13 | — | — | — | turbid solution | — |
| 26 | — | — | — | turbid solution | — |

TABLE 4
Stability of the preparation according to Example 4 (batch 8591)

| | | | | Appearance of | |
|---|---|---|---|---|---|
| Example 4 | | HPLC- | | lyophilisate after | |
| Storage | | SEC | | dissolution in | |
| time | Protein | EMD | KSA- | 4.0 ml of bidist. | Turbidity |
| (weeks) | determination | 273066 | ELISA | water | by photometry |
| [weeks] | [mg/ml] | [%] | [mg/ml] | dissolved | A550 |
| Lyophilisate | 0.849 | 96.14 | — | clear colourless | — |
| after prepn. | | | | solution | |

Storage at 40° C./75% r.h.

| | | | | | |
|---|---|---|---|---|---|
| 14 | — | 94.42 | 4.13 | clear colourless | — |
| | | | | solution | |

1. Lyophilised pharmaceutical preparation of immunocytokines, comprising an immunocytokine, a sugar or an amino sugar, an amino acid and a surfactant.

2. Lyophilised pharmaceutical preparation according to claim 1, characterised in that the immunocytokine present is an immunocytokine which contains, as cytokine constituent, cytokines selected from the group consisting of cytokines which have, as common structural feature, a bundle of four α-helices.

3. Lyophilised pharmaceutical preparation according to claim 2, characterised in that the immunocytokine contains, as cytokine constituent, an interleukin, an interferon and/or a haematopoietic growth factor.

4. Lyophilised pharmaceutical preparation according to claim 3, characterised in that the immunocytokine contains, as cytokine constituent, interleukin-2 (IL-2).

5. Lyophilised pharmaceutical preparation according to claim 1, characterised in that it essentially consists of an immunocytokine, a sugar or amino sugar, an amino acid, a buffer and a surfactant.

6. Lyophilised pharmaceutical preparation according to claim 1, characterised in that the sugar is a mono-, di- or trisaccharide, preferably sucrose, lactose, maltose or trehalose.

7. Lyophilised pharmaceutical preparation according to claim 1, characterised in that the amino sugar is glucosamine, N-methylglucosamine, galactosamine or neuraminic acid.

8. Lyophilised pharmaceutical preparation according to claim 1, characterised in that the amino acid is a basic, acidic or neutral amino acid, preferably arginine, lysine or omithine.

9. Lyophilised pharmaceutical preparation according to claim 1, characterised in that the surfactant is a nonionic surfactant.

10. Lyophilised pharmaceutical preparation according to claim 9, characterised in that the surfactant is a polysorbate or a polyoxyethylene-polyoxypropylene polymer.

11. Lyophilised pharmaceutical preparation according to claim 10, characterised in characterised in that the surfactant is the polyoxyethylene sorbitan fatty acid ester polyoxyethylene (20) sorbitan monooleate or polyoxyethylene (20) sorbitan monolaurate.

12. Lyophilised pharmaceutical preparation according to claim 1, characterised in that an isotonic agent is furthermore present in an amount necessary for establishing isotonicity.

13. Aqueous pharmaceutical preparation of immunocytokines which is obtainable by reconstitution of the lyophilisate according to claim 1 with an aqueous solvent.

14. Aqueous pharmaceutical preparation according to claim 13, characterised in that the solution has a pH of 5-8, preferably 5.6-7.4.

15. Aqueous pharmaceutical preparation according to claim 14, characterised in that the solution has a pH of 6-7.

16. Process for the preparation of a lyophilised pharmaceutical preparation according to claim 1, characterised in that an aqueous preparation comprising an immunocytokine, a sugar or amino sugar, an amino acid, a surfactant and, if desired, further pharmaceutical adjuvants is prepared, and the solution is subsequently lyophilised.
